(19) United States
(12) Patent Application Publication
(10) Pub. No.: US 2012/0070908 A1
(43) Pub. Date: Mar. 22, 2012

(54) IMMUNO CONJUGATE AND PROCESS FOR PREPARATION THEREOF

(75) Inventors: Chander Raman SURI, Chandigarh (IN); Jas Deep Kaur, Chandigarh (IN); Kanwar Vikas Singh, Chandigarh (IN); Grish Chandra Varshney, Chandigarh (IN); Manoj Raje, Chandigarh (IN)

(73) Assignee: COUNCIL OF SCIENTIFIC AND INDUSTRIAL RESEARCH, New Delhi (IN)

(21) Appl. No.: 13/087,024

(22) Filed: Apr. 14, 2011

Related U.S. Application Data
(63) Continuation of application No. 11/252,621, filed on Oct. 19, 2005, now abandoned.

(30) Foreign Application Priority Data
Jul. 26, 2005 (IN) 1983/DEL/2005

(51) Int. Cl. G01N 21/78; G01N 21/55; G01N 33/53; B82Y 15/00

(52) U.S. Cl. 436/501; 977/810; 977/902

(57) ABSTRACT

Present invention deals with a new immuno-reagent comprising hapten (MPAD)-protein-gold for pesticides detection. The conjugate MPAD-protein-gold competes with the analyte of interest for a finite number of binding sites provided by anti-atrazine antibodies coated on nitrocellulose membrane. The newly developed conjugate has a long shelf life with high stability at 4° C. The dynamic concentration range for standard atrazine solutions shows a linear inhibition (decrease in intensity of color) between 10 ppb to 1 ppm atrazine in water samples.

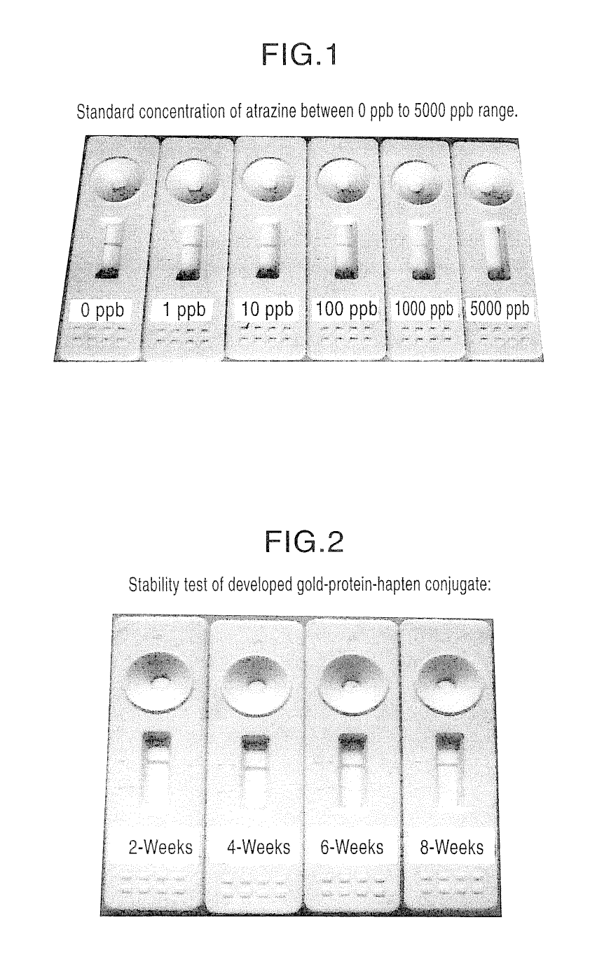

### FIELD OF INVENTION

The present invention relates to a novel gold-protein-hapten conjugate and process for the preparation of said novel conjugate.

More particularly, it relates to a kit for rapid analyzing a sample for pesticides monitoring using a novel gold-protein-hapten conjugate.

### BACKGROUND AND PRIOR ART

Pesticides, derived from chemicals, are an essential input in increasing agricultural production by preventing crop losses before and after harvesting. However, their indiscriminate use, apart from being an operational hazard, is posing a serious threat to human health. These organic toxins enter animals and human beings directly or indirectly through the food chain or drinking water. These chemicals being degraded slowly leave behind residues in food and water sources and concentrate as they move up the food chain. Because of their severe toxicity, even at trace levels, it is essential to monitor the levels of these pesticides in the environment, food stuffs, and soil. Due to their recalcitrant and toxic nature, they are listed among priority pollutants. The quick detection of these pollutants is of vital importance for the environmental cleanup.

Dipstick based rapid detection of these pollutants has been reported earlier (Giersch, T., J. Agric. Food Chem. 1993, 41: 1006-1011; Mosiello, L. et al., J. Agric. Food Chem. 1998, 46: 3847-3851; Heiss, C, Weller, MG and Niessner, R., Anal. Chim. Acta 1999, 396: 309-316) wherein, Giersch, T. (J. Agric. Food Chem. 1993, 41: 1006-1011) developed a dipstick-based immunoassay using nitrocellulose membrane spotted with goat anti-mouse IgG antibody. In this approach, the strips were treated with specific anti-atrazine antibody (monoclonal antibody, K4E7). The standard atrazine and tracer (atrazine labeled with horseradish peroxidase) solutions were added onto the antibody spots. After washing the membrane with phosphate buffer, the color was developed by treating the strips with tetramethylbenzidine (TMB) substrate. This approach is basically a simple dot-blot based immunoassay where antibody-antigen complex are formed in a solid phase. This approach requires quite a large assay time (5-6 hours) for the complete assay. Similarly, the work of Mosiello, Let al., (J. Agric. Food Chem. 1998, 46: 3847-3851) and Heiss, C, Weller, M G and Niessner, R. (Anal. Chim. Acta 1999, 396: 309-316) also describe the same approach of making dipstick assay in the stationary phase. The drawbacks of the reported methods are that these approaches require very long time for assay (more than 7-8 hours per assay), and also use immuno-reagents, which are not very stable for long-term usages.

### OBJECT OF THE INVENTION

The main object of the present invention is to provide a novel gold-protein-hapten conjugate.

Another object of the present invention is to provide a process for preparing a said novel conjugate.

Yet object of the present invention is to provide a kit of rapid detection of pesticide level from liquid sample by using a novel conjugate.

Still another object of the present invention is to provide a method for detecting and measuring pesticides from water sample by using the said kit.

### BRIEF DESCRIPTION OF THE PHOTOGRAPHS

In the photograph(s) accompanying this specification Photograph 1 represents the standard concentration of atrazine in the range between 0 ppb to 1 ppm.

Photograph 2 represents the stability test of developed gold-protein-hapten conjugate

### SUMMARY OF THE INVENTION

The Present invention relates to a kit, based on lateral flow principle where anti pesticide antibody is immobilized at the detection zone on the nitrocellulose membrane while another antibody is coated nearby. The amount of free pesticide present in the water sample competes with the gold protein hapten conjugate to bind with the available limited antibodies binding sites. The color developed due to immuno conjugate is correlated with the concentration of sample. The method is quite simple and specific for the target compound atrazine). The novel gold protein hapten conjugate is highly stable under storage condition at 4° C.

### DETAILED DESCRIPTION OF THE INVENTION

The present invention is based on the lateral flow of immunoconjugate on a dipstick membrane, is rapid and easy to use for pesticides screening. The conjugate (gold-protein-hapten), which is used as detector reagent in the present invention, is around two fold more stable than the existing protein-gold conjugate used for dipstick applications. The present assay could also be useful in the detection of other toxic compounds such as drugs, heavy metals etc. by using specific anti-analyte antibodies.

The stepwise details of the present invention are: preparation of a novel conjugate, coating of antibodies on nitrocellulose membrane using standard techniques, development of dipstick format using the conjugate as prepared in step 1, and using a portable reflectometric scanner for semi-quantification of pesticide concentration in sample.

The details of the present invention involve development of a new reagent immunoconjugate for the detection purpose in a dipstick based immunoassay format. For this, a derivative of target pesticide atrazine (mercaptopropionic acid derivative of atrazine) was first synthesized by using standard procedure as reported earlier [K V Singh et al., (2003) J. Anal. Bioanal. Chem.] This was done by adding slowly under constant stirring, a solution of 3-mercaptopropionic acid (5.5 mmole) and 85% KOH (10.8 mmole) made in 10 ml ethanol to a solution of 5.01 mmol atrazine made in 50 ml ethanol. The mixture was refluxed for 6 hours and then the solvent was evaporated under reduced pressure. The residue was taken up in 25 ml 5% NaHCO₃ and washed three times with chloroform. The aqueous layer of the solution was acidified with 6N HCl causing the acidic derivative to precipitate immediately. The supernatant was decanted and the derivative was dried under mild vacuum. The precipitate was further dissolved in ethanol, and then allowed under reduced pressure at 37° C. to form mercaptopropionic acid derivative crystals (MPAD). Conjugates of atrazine derivative with protein-gold complex were prepared by mixing protein solution and mercaptopropionic acid derivative of atrazine made in 1 ml dimethylformamide (DMF) along with 125 μmol of dicyclohexyl carbodiimide (DCC) and 125 μmol of N-hydroxysuccinimidyl ester (NHS). The mixture was incubated for 4 h and then centrifuged to remove the urea precipitate. The complex was formed by incubating the protein in the presence of marcaptoethylamine overnight at 4° C., which breaks di-sulfide bonds of the protein so as to link with gold particles to provide a stable complex. The conjugate developed was used in the dipstick format using a nitrocellulose membrane on which anti-pesticide antibody is immobilized at the test line while another antibody (anti-ovalbumin antibody) at the control line on the detection zone. The sample is introduced through the sample pad affixed at one end of the nitrocellulose membrane while on the other end; an absorbance pad is attached to increase the flow of molecules onto the membrane. The sample along with the conjugate move onto the nitrocellulose membrane where these two different molecules react competitively to the available binding sites of the anti-atrazine antibodies coated on the membrane. The intensity of color developed (reversibly) give the presence of analyte in the sample. This was further semi-quantified using a small hand-held reflectometer scanner.

Accordingly the present invention provides a process for preparation of a novel immuno conjugate comprising

(a) providing a mercaptopropionic acid derivative of atrazine (Hapten) by known method,

(b) Chemisorbing the colloidal gold particles of about 20 nm in size to a reduced carrier protein through its thiol groups to obtain the gold protein complex,

(c) mixing the complex obtained from step (b) with hapten along with dicyclohexyl carbodiimide (DCC) and N-hydroxysuccinimidyl ester (NHS) dissolved in the solvent Dimethylformamide (DMF),

(d) incubating the mixture obtained from step (c) followed by centrifuging to remove the urea precipitate and to obtain the desired immuno conjugate process for preparation of a novel immuno conjugate.

In an embodiment of the present invention, an atrazine derivative hapten is synthesized by known method as described in [K V Singh et al., (2003) J. Anal bioanal. Chem.] In another embodiment of the present invention, the solution of colloidal gold particles are prepared by heating of about 200 ml of 0.01% gold chloride solution to the boiling point. In yet another embodiment of the present invention, about 4 ml of 1% of tri sodium citrate is added in the boiling solution of gold chloride.

In still another embodiment of the present invention, the color of the gold chloride solution is turned immediately to dark purple indicating formation of colloidal gold solution.

In still another embodiment of the present invention, the UV visible spectra showed a peak of colloidal gold solution at 525 nm.

In still another embodiment of the present invention, the colloidal gold is chemosorbed to the carrier protein (Ovalbumin) by mixing the reduced ovalbumin to Hapten (mercaptopropionic acid derivative of atrazine).

In still another embodiment of the present invention, the reduced carrier protein solution is formed by incubating the ovalbumin with marcaptoethylamine overnight at about 4° C. to break the di-sulfide bonds of the protein so as to link with colloidal gold particles (20 nm) to provide stable gold- protein complex.

In still another embodiment of the present invention, the hapten is covalently linked to the protein-gold complex via lysine groups.

In still another embodiment of the present invention, the coupling agents used are dicyclohexyl carbodiimide (DCC) and N- hydroxysuccinimidyl ester (NHS) in ratio of 1:1 dissolved in solvent Dimethylformamide (DMF). at room temperature for about 4 hours.

In still another embodiment of the present invention the mixture is centrifuged at about 10000 rpm for about 5 min to remove the urea precipitate and to obtain a novel immuno conjugate.

Further, the present invention also provides a novel immuno conjugate.

In an embodiment of the present invention, the said immuno conjugate comprises gold, protein hapten in the ratio ranging between 1:25:0.48-1:26:0.5.

In another embodiment of the present invention, the hapten used is mercaptopropionic acid derivatives of atrazine.

In yet another embodiment of the present invention, the protein used is ovalbumin having stock radius about 5 nm.

In still another embodiment of the present invention, the number of protein molecules per gold particle is ranging between 25-26 by taking protein cover ap. 50% surface area.

In still another embodiment of the present invention, the number of hapten per carrier protein molecule is ranging between 12-13 by taking hapten-protein molar ratio 1:50.

In still another embodiment of the present invention, the said colloidal gold size is about 20 nm.

In still another embodiment of the present invention, the number of colloidal gold particles per ml are App. 5×10¹².

In still another embodiment of the present invention, the available surface area on 20 nm colloidal gold particles is 1256 nm².

Present invention also provides a kit for rapid detection of pesticide in a water sample comprising at least one test line, a control line immobilized in detection zone present on or within a support and an immuno conjugate.

In another embodiment of the present invention, an anti ovalbumin is immobilized in a detection zone present on or within a support i.e. nitrocellulose membrane.

In yet another embodiment of the present invention, the water sample contaminated with atrazine is introduced through the sample pad affixed at one end of the nitrocellulose membrane.

In still another embodiment of the present invention, an absorbance pad is attached to increase the flow of molecules onto the membrane.

In still another embodiment of the present invention, the detection of atrazine (pesticide present in water sample) is based on competitive binding of specific anti atrazine antibodies to the immunoconjugate and atrazine sample.

In still another embodiment of the present invention, the atrazine is semi quantified with the help of a small hand held reflectometer.

The present invention further provides a method for detecting and measuring the pesticide from a water a sample.

In an embodiment of the present invention, 50 μl of water sample is taken in the sample well.

In another embodiment of the present invention, 50 μl of gold protein hapten conjugate is added in the water sample.

In yet another embodiment of the present invention, the sample along with the conjugate move onto the nitrocellulose membrane where these two different moluecules react competitively to the available binding sites of the anti atrazine antibodies coated on the membrane.

In still another embodiment of the present invention, the wine red color is generated on the test line (coated with anti atrazine antibodies) and control line (coated with anti ovalbumin antibodies) indicating the presence of pestiside (atrazine).

In still another embodiment of the present invention the colour is detected visually indicating the presence and amount of the pesticide.

In still another embodiment of the present invention a calibration curve in the form of visual presentation is formed which shows the pesticide level in the water sample.

The color intensity of developed hapten-protein-gold conjugate did not show significant loss of intensity even after 6 weeks of its storage time at ambient temperature. This is mainly because of the reason stronger bondformation between gold protein complex made by chemosorbing colloidal gold particles to the reduced albumin through strong Au-S bonds. So this dipstick could successfully be used for around 6-8 weeks time under normal ambient conditions.

The following examples are given by way of illustration and therefore should not be construed to limit the scope of the present invention.

### Example 1

The immuno-conjugate comprising hapten, protein (ovalbumin) and colloidal gold particles were developed for the detection of atrazine in water samples using dipstick based immunoassay format. For this, a solution of colloidal gold particles (20 nm in size) was prepared by heating 200 ml of 0.01% gold chloride solution to boiling point. In the boiling solution of gold chloride, 4 ml of 1% trisodium citrate was quickly added. The color of the solution turned immediately to dark purple indicating formation of colloidal gold. UV-visible spectra showed a peak at 525 nm. The colloidal gold was chemisorbed to the carrier protein (ovalbumin) by mixing the reduced ovalbumin to hapten (mercaptopropionic acid derivative of atrazine). The atrazine derivative was synthesized as described earlier [K V Singh et al., (2003) J. Anal. Bioanal. Chem]. Conjugates of this derivative with protein-gold complex were prepared by mixing protein solution and atrazine derivative of atrazine made in 1 ml dimethylformamide along with 125 μmol of dicyclohexyl carbodiimide and 125 μmol of N-hydroxysuccinimidyl ester. The mixture was incubated for 4 h at room temperature and then centrifuged for 5 min at 10000 RPM to remove the urea precipitate. The gold-protein complex was formed by incubating the protein in the presence of marcaptoethylamine overnight at 4° C., which breaks di-sulfide bonds of the protein so as to link with colloidal gold particles (20 nm in size) to provide stable gold-protein complex.

### Example 2

The development of dipstick format for monitoring pesticides concentration:

The assay is based on the competitive binding of specific anti-atrazine antibodies (bound to nitrocellulose membrane) to the fixed amount of hapten-protein-gold conjugate and atrazine sample. The assay is performed by the application of 50 μl of water sample in the sample well of the dipstick device followed by the addition of 50 μl of above conjugate. The color (wine red) generated on the test line (coated with anti-atrazine antibodies) and control line (coated with anti-ovalbumin antibodies) indicated the presence of atrazine in sample. A hand-held reflectometer scanner at 657 nm was used for the semi-quantification of the test samples. Calibration curve was obtained with the standard atrazine solutions (0 ppb to 5000 ppb), which was further, used for the correlation with atrazine concentration in water sample. The developed approach is very fast and could be very useful for field monitoring.

### ADVANTAGES

The main advantages of the present invention are:

1. Use of stable immuno-reagents for the fast detection of pesticides. Increased shelf life of a novel immunoconjugate (protein-gold hapten conjugate) means did not show any loss in its activity and sensitivity even after three months of its storage at 4° C.

2. Rapid screening of pesticide samples.

3. Semi-quantification of pesticides present in the sample is possible using a less complicated hand-held reflectometer scanner.

1.-21. (canceled)

22. A method for detecting a pesticide in a sample comprising:
providing a kit comprising at least one test line comprising an anti-pesticide antibody and at least one control line comprising an anti-carrier protein antibody, wherein the at least one test line and the at least one control line are immobilized in a detection zone;
contacting the kit with a sample and a conjugate comprising gold, a carrier protein, and a pesticide derivative;
and detecting the presence of the pesticide in the sample.

23. The method of claim 22, wherein the contacting of the sample with the test line generates color in the test line.

24. The method of claim 23, wherein the color generated is wine red.

25. The method of claim 23, wherein the color is detected visually indicating the presence of the pesticide.

26. The method of claim 22, wherein a calibration curve is provided using standard pesticide solutions ranging from 0 ppb to 5000 ppb.

27. The method of claim 22, wherein the at least one test line and the at least one control line are provided on a support comprising nitrocellulose.

28. The method of claim 22, wherein the pesticide comprises atrazine

29. The method of claim 22, wherein the detection is based on competitive binding of anti-atrazine antibodies to the immunoconjugate and the sample.

30. The method of claim 22, where the immuno conjugate comprises gold particle:carrier protein:pesticide derivative in the ratio ranging between 1:25:0.48 and 1:26:0.5.

31. The method of claim 22, wherein the pesticide derivative comprises mercaptopropionic acid derivatives of atrazine.

32. The method of claim 22, wherein carrier protein comprises ovalbumin.

33. The method of claim 30, wherein number of carrier protein molecules per gold particle is between 25 and 26.

34. The method of claim 30, wherein number of pesticide derivative molecules per carrier protein molecule is between 12 and 13.

35. The method of claim 30, wherein the size of the gold particles is about 20 nm.

36. The method of claim 30, wherein number of gold particles per ml of conjugate is about 5×10¹².

37. The method of claim 35, wherein available surface area on 20 nm gold particles is about 1256 nm².

38. The method of claim 22, wherein the test line comprises an anti-atrazine antibody.

39. The method of claim 22, wherein the control line comprises an anti-ovalbumin antibody.

40. The method of claim 26, further comprising quantifying the pesticide via a reflectometer scanner.
